(19) United States
(12) Patent Application Publication
(10) Pub. No.: US 2014/0018836 A1
(43) Pub. Date: Jan. 16, 2014

(54) Endo-Safe-Bag-Gasless support system

(71) Applicants: Yen-Ming Yeh, New Taipei City (TW); Kuei-Huang Wu, Taoyuan (TW)

(72) Inventors: Yen-Ming Yeh, New Taipei City (TW); Kuei-Huang Wu, Taoyuan (TW)

(73) Assignee: TOP-BOUND ENTERPRISE CO., LTD, New Taipei City (TW)

(21) Appl. No.: 13/548,204

(22) Filed: Jul. 13, 2012

(51) Int. Cl. A61M 29/02

(52) U.S. Cl. 606/192

(57) ABSTRACT

The present invention relates to an Endo-Safe-Bag-Gasless support system, comprising: an air tube, having a gas port formed on the tube side thereof; at least two inflatable bags, which are connected to the air tube and have one laminating-line layer on the surface thereof; and a plurality of air bags, which is formed on the laminating-line layer.

### BACKGROUND OF THE INVENTION

1. Technical Field

The present invention relates to a inflatable bag, and more particularly to an Endo-Safe-Bag-Gasless support system which is used in surgical operation for being dived into and expand the body cavity.

2. Description of Related Art

Minimally invasive surgery is an important development in medical technology, wherein when operating the minimally invasive surgery to a patient, it merely causes minor trauma and small wound on the patient's body. So that, compared with the traditional surgery, the minimally invasive surgery includes the advantages of small incision, less trauma, rapid recovery and less pain for patients, and such advantages of the minimally invasive surgery also bring the revolution for modern medical technology. In minimally invasive surgery treatment, the body cavity of a target organ must be opened with a cavity opening for inserting surgical instruments. In addition, because the body cavity only includes the limited cavity space, the surgeon would inflate gas into the body cavity for expanding the body cavity when operating the surgery. However, such surgery way includes the shortcomings and drawbacks as follows:

1. The patients would have flatulence and feel uncomfortable after surgery because the gas inflated into the body cavity cannot be shed, and the worst of all, the gas inflated into the body cavity would compress the internal organs, resulting in damage of internal organs.
2. The gas inflated into the body cavity must be the specific gas for avoiding the blood is combined with the gas, resulting in adverse effect. Thus the ease of the surgery is affected because the limited usage of the gas.

Accordingly, in view of the shortcoming of the conventional stylus, the inventor of the present application has made great efforts to make inventive research thereon and eventually provided an Endo-Safe-Bag-Gasless support system.

### BRIEF SUMMARY OF THE INVENTION

The first objective of the present invention is to provide an Endo-Safe-Bag-Gasless support system for avoiding the gas from being directly inflated into the body in order to expand the body cavity in surgery, therefore the flatulence after surgery can be effectively prevented.

The second objective of the present invention is to provide an Endo-Safe-Bag-Gasless support system for expanding the body cavity by an inflatable bag in surgery, wherein the inflatable bag can be inflated by using any kinds of gas, and such feature brings a big convenience to the surgeon when the surgery is operated.

Accordingly, for achieving the above objectives of the present invention, the inventors propose an Endo-Safe-Bag-Gasless support system, comprising:

an air tube, having a gas port formed on the tube side thereof;

at least two inflatable bags, connected to the air tube, and each inflatable bag has a laminating-line layer on the surface thereof, used for withstanding the pressure;

a plurality of air bags, formed on the laminating-line layer;

wherein an external gas can be inflated into the gas port, and the gas would be further inflated into the inflatable bags via the air tube, so as to make the inflatable bag be full of the gas.

### BRIEF DESCRIPTION OF THE SEVERAL VIEWS OF THE DRAWINGS

The invention as well as a preferred mode of use and advantages thereof will be best understood by referring to the following detailed description of an illustrative embodiment in conjunction with the accompanying drawings, wherein:

FIG. 1 is the first appearance diagram of the Endo-Safe-Bag-Gasless support system according to the present invention;

FIG. 2 is a appearance diagram of the stylus structure according to the present invention; and

FIG. 3 is a cross-section diagram of the Endo-Safe-Bag-Gasless support system according to the present invention.

### DETAILED DESCRIPTION OF THE INVENTION

To more clearly describe a stylus structure according to the present invention, embodiments of the present invention will be described in detail with reference to the attached drawings hereinafter.

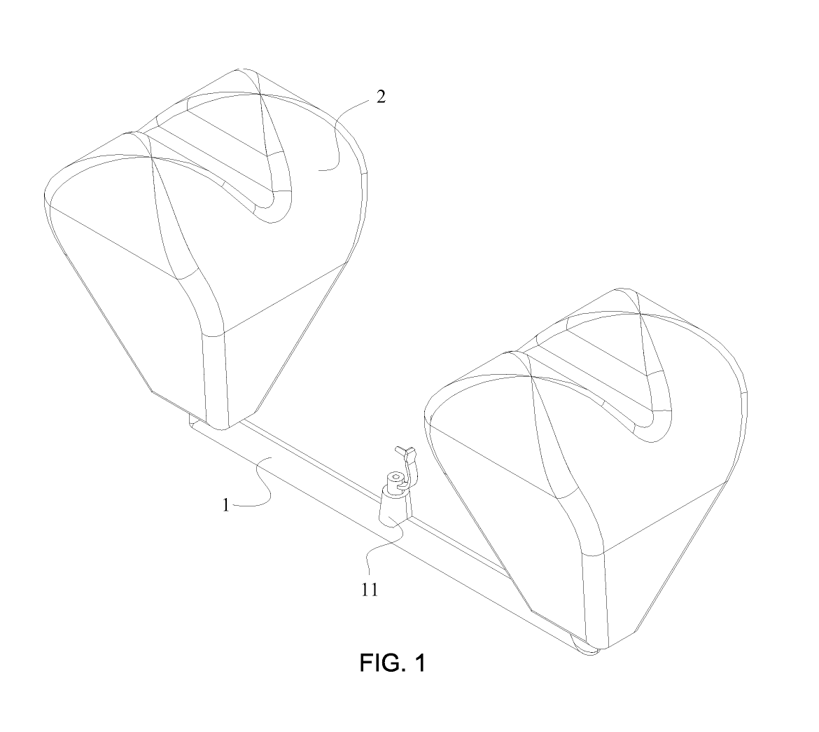

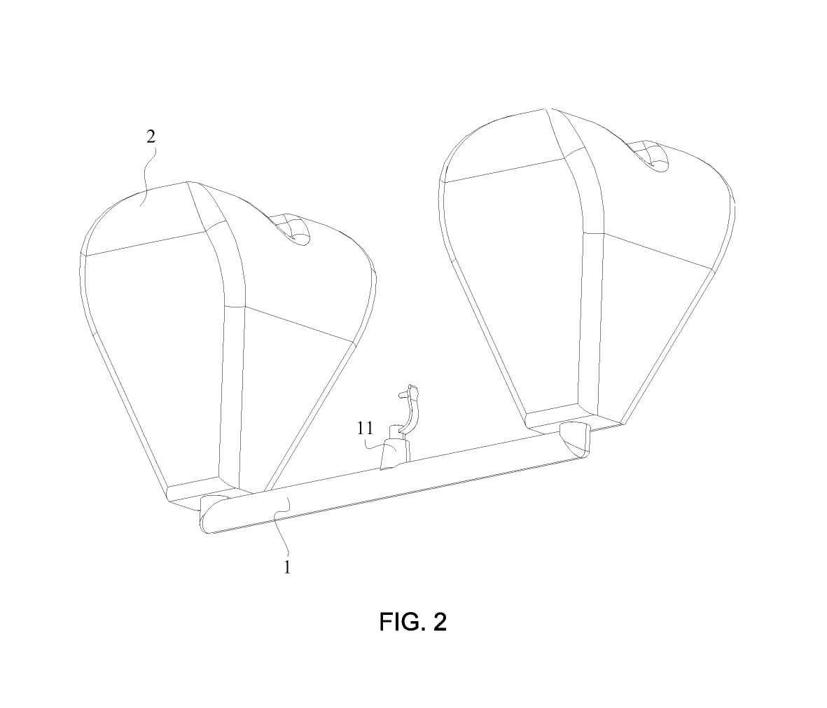

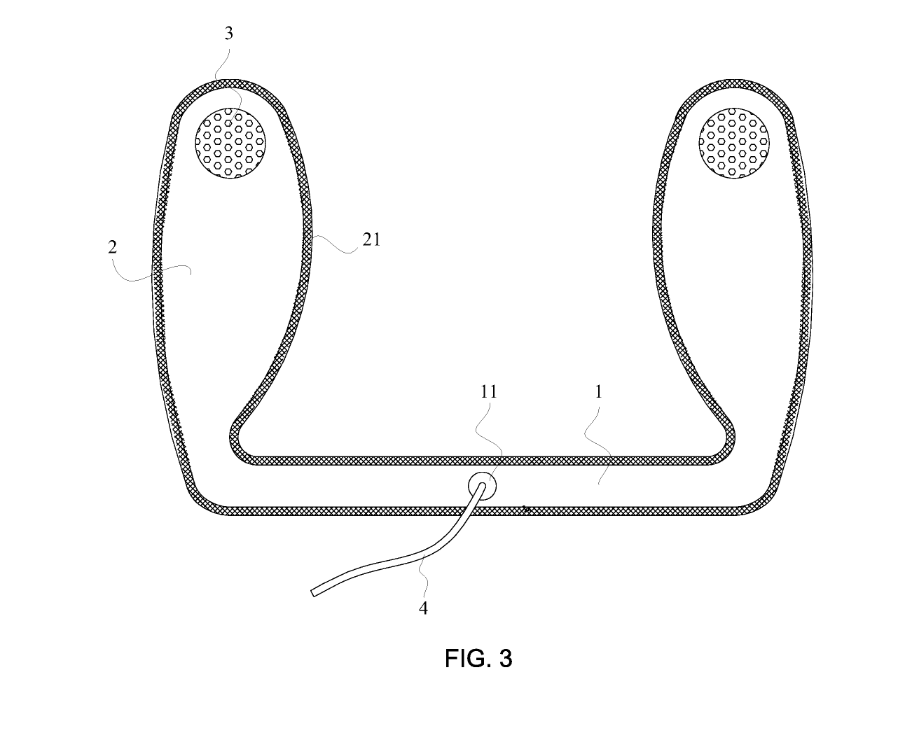

With reference to FIGS. 1-3, there are shown the first appearance diagram, the second appearance diagram, and a cross-section diagram of the Endo-Safe-Bag-Gasless support system according to the present invention, comprising:

An air tube 1, having a gas port 11 formed on the tube side thereof, wherein the gas tube 11 is made of PVC(polyvinyl chloride), and being connected to a inflatable device 4 for inflating the gas into the air tube 1; the gas port 11 further comprises a gas check valve for preventing gas backflow; two inflatable bags 2, being connected to the air tube 1, and each inflatable bag 2 having a laminating-line layer 21 on the surface thereof, used for withstanding at least 720 mmHG pressure, wherein the thickness of the laminating-line layer 21 is ranged from 3 mm to 4 mm; and a plurality of air bag 3, being formed on the laminating-line layer 21.

Thus, through the embodiment, the Endo-Safe-Bag-Gasless support system of the present invention has been completely and clearly disclosed in the above description. The following description is about the detail of the operation method. The two inflatable bags 2 are not inflated before surgery. In the surgery process, the doctor would open a small hole on the body cavity of the target organ, and insert the inflatable bags 2 via the hole into the body cavity, and then inflate the gas into the inflatable bag 2 for expanding the body cavity by the inflatable device 4.

After the surgery, the gas in the inflatable bag 2 would be led out, and then the inflatable bags 2 would be taken out of the body cavity. Then the wound is sutured to complete the surgery. Thus, through the above mentioned method, it is not necessary for patients to inflate the gas into the cavity directly, causing the flatulence.

In the end, the present invention has been completely and clearly disclosed in the above description, and in summary, the present invention has the following advantages:

1. To avoid the gas is inflated directly into the body to expand the body cavity in surgery, the inflatable bags are applied to set in the body cavity and inflated to expand the body cavity for preventing the flatulence or damage by oppressing visceral after surgery.
2. The inflatable bags could be inflated in any kinds of gas and the gas could be recycled, so as to avoid the body cavity must inflated in the specific gas.

The above description is made on embodiments of the present invention. However, the embodiments are not intended to limit scope of the present invention, and all equivalent implementations or alterations within the spirit of the present invention still fall within the scope of the present invention.

We claim:

1. An Endo-Safe-Bag-Gasless support system, comprising:
an air tube, having a gas port formed on the tube side thereof;
at least two inflatable bags, being connected to the air tube, and each inflatable bag having a laminating-line layer on the surface thereof, used for withstanding the pressure;
a plurality of air bags, being formed on the laminating-line layer;
wherein an external gas can be inflated into the gas port, and the gas would be further inflated into the inflatable bags via the air tube, so as to make the inflatable bag be full of the gas.

2. The Endo-Safe-Bag-Gasless support system of claim 1, wherein an inflating device can be connected to the gas port for inflating the gas into the air tube.

3. The Endo-Safe-Bag-Gasless support system of claim 1, wherein the thickness of the laminating-line layer is ranged from 3 mm to 4 mm.

4. The Endo-Safe-Bag-Gasless support system of claim 1, wherein the laminating-line layer is capable of withstanding at least 720 mmHG pressure.

5. The Endo-Safe-Bag-Gasless support system of claim 1, wherein the gas port is made of PVC (polyvinyl chloride).
